(19) United States
(12) Patent Application Publication
(10) Pub. No.: US 2013/0149266 A1
(43) Pub. Date: Jun. 13, 2013

(54) Long lasting fingernail lacquer and method for manufacture

(71) Applicants: Michael Mitsuo Homma, Katy, TX (US); Victor Masaru Homma, West Valley City, UT (US)

(72) Inventors: Michael Mitsuo Homma, Katy, TX (US); Victor Masaru Homma, West Valley City, UT (US)

(21) Appl. No.: 13/694,527

(22) Filed: Dec. 10, 2012

Related U.S. Application Data
(60) Provisional application No. 61/569,017, filed on Dec. 9, 2011.

(51) Int. Cl. A61K 8/73; A61K 8/81

(52) U.S. Cl. CPC A61K 8/731; A61K 8/81
USPC 424/61

(57) ABSTRACT

The invention provides long lasting and quick drying nail polish compositions for application to natural or artificial nails. The nail polish composition comprises a lacquer component and an optional pigment component. The lacquer component can harden in ambient atmosphere without the use of ultraviolet radiation. The lacquer component includes acrylic polymers dissolved in organic solvents.

### CROSS-REFERENCE TO RELATED APPLICATION

This application claims the benefit of U.S. Provisional Patent Application No. 61/569017 filed on Dec. 9, 2011, which is incorporated herein by reference.

### BACKGROUND OF INVENTION

1. Field of Invention

The present invention relates generally to films which are useful as cosmetic agents or therapeutic agents as well as methods of their use. More particularly, the present invention relates to films which, when applied to mammalian nails exhibit extremely long wear, leaves a very shinny surface. can be removed with ordinary fingernail polish remover quickly, dries very fast, and does not require UV light to cure. This is something that can be done at home and doesn't require a trip to the nail salon.

2. Description of the Related Art

In the field of fingernail lacquers and fingernail coatings. Prior Art inventions met one or more of the above requirements but not all. For example, the prior art would have a glossy shine but would dry very slowly. Other prior art such as UV Gels would dry quickly with a high gloss and be very long lasting, but would not come off without soaking in acetone for up to 10 minutes or mechanically grinding off the coating and require exposing the client to a UV light in order to cure the Gel. Prior art that was fast drying would not last long and the shine would be much less glossy than the UV Gels or slower drying high gloss coatings. The unique combination of quick drying able to be removed with common acetone based nail polish remover, very long lasting, does not require UV light to harden, and had a very glossy shine was not achievable with any of the prior art embodiments. Previous coatings included standard nitrocellulose based nail lacquers. These lacquers dry very slowly and do not last very long, but can leave a very glossy finish. These formulations typically contain nitrocellulose, acrylic co-polymers, iso-propyl alcohol, ethyl acetate, butyl acetate, plasticizer agents and viscosity modifiers.

Previous coatings included standard nitrocellulose based nail lacquers. These lacquers dry very slowly and do not last very long, but can leave a very glossy finish. These formulations typically contain nitrocellulose, acrylic co-polymers, iso-propyl alcohol, ethyl acetate, butyl acetate, plasticizer agents and viscosity modifiers.

Previous coatings included standard nitrocellulose based nail lacquers. These lacquers dry very slowly and do not last very long, but can leave a very glossy finish. These formulations typically contain nitrocellulose, acrylic co-polymers, iso-propyl alcohol, ethyl acetate, butyl acetate, plasticizer agents and viscosity modifiers.

Previous coatings included fast drying fingernail lacquers and polishes which sacrifice high gloss and longer lasting for quick drying. Typical formulations consist of higher concentrations of quick drying solvents such as ethyl acetate and less of an acrylic co-polymer component. These modifications cause the coating to dry faster, but without these ingredients, the coating is less shinny and does not have long lasting behavior.

Thus, there is a need for a quick drying, long lasting fingernail coating that has a high level of shine, does not chip or come off easily, and can be removed with ordinary fingernail polish remover in a short period of time.

### SUMMARY OF INVENTION

The invention provides a composition comprising an acrylic polymer in an organic solvent. The composition can be added to the current nail polish compositions, wherein the final composition comprises about 0.5% to about 10% (w/w) of the acrylic polymer. The inventive compositions can be applied to fingernails.

The invention is directed to new formulations for fingernail lacquer and fingernail topcoats where combining a solution of Elvacite 2552? acrylic co-polymer and ethyl acetate to standard high gloss fingernail topcoat or fingernail lacquer to create a mixture that contains from about 0.5 to about 10 weight percent of the acrylic co-polymer. The addition of the acrylic co-polymer creates a new product that is fast drying and has much higher durability than the original fingernail topcoat or fingernail lacquer, is very shiny and is easily removed with standard acetone or non-acetone based fingernail polish remover.

In one aspect, the invention provides a nail polish comprising about 0.5% to about 10% of an acrylic polymer dissolved in an organic solvent, about 4% to about 20% by weight of a film-forming polymer, wherein the film-forming polymer is nitrocellulose, cellulose acetate, cellulose acetate-butyrate, ethyl cellulose, polyvinyl acetate, polyvinyl butyrate, or combinations thereof, about 1% to about 4% by weight of a plasticizer, and about 10% to about 50% by weight solvent system.

### DETAILED DESCRIPTION OF THE INVENTION

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. Definition of standard chemistry terms may be found in reference works, including Carey and Sundberg (2004) “Advanced Organic Chemistry 4^{rd} Ed.” Vols. A and B, Springer, N.Y. The practice of the present invention will employ, unless otherwise indicated, conventional methods of mass spectroscopy, protein chemistry, biochemistry, and pharmacology, within the skill of the art.

The term “alkyl” means the monovalent branched or unbranched saturated hydrocarbon radical, consisting solely of carbon and hydrogen atoms, having from one to twelve carbon atoms inclusive, unless otherwise indicated. Examples of alkyl radicals include, but are not limited to, methyl, ethyl, propyl, isopropyl, butyl, isobutyl, sec-butyl, tert-butyl, pentyl, n-hexyl, octyl, dodecyl, and the like.

The term “alkylene” as used herein means the divalent linear or branched saturated hydrocarbon radical, consisting solely of carbon and hydrogen atoms, having from one to eight carbon atoms inclusive, unless otherwise indicated. Examples of alkylene radicals include, but are not limited to, methylene, ethylene, trimethylene, propylene, tetramethylene, pentamethylene, ethylethylene, and the like.

The term “alkenylene” means the divalent linear or branched unsaturated hydrocarbon radical, containing at least one double bond and having from two to eight carbon atoms inclusive, unless otherwise indicated. The alkenylene radical includes the cis or trans ((E) or (Z)) isomeric groups or mixtures thereof generated by the asymmetric carbons. Examples of alkenylene radicals include, but are not limited to ethenylene, 2-propenylene, 1-propenylene, 2-butenyl, 2-pentenylene, and the like.

The term “aryl” means the monovalent monocyclic aromatic hydrocarbon radical consisting of one or more fused rings in which at least one ring is aromatic in nature, which can optionally be substituted with hydroxy, cyano, lower alkyl, lower alkoxy, thioalkyl, halogen, haloalkyl, hydroxyalkyl, nitro, alkoxycarbonyl, amino, alkylamino, dialkylamino, aminocarbonyl, carbonylamino, aminosulfonyl, sulfonylamino, and/or trifluoromethyl, unless otherwise indicated. Examples of aryl radicals include, but are not limited to, phenyl, naphthyl, biphenyl, indanyl, anthraquinolyl, and the like.

The term “halogen” as used herein refers to fluoro, bromo, chloro and/or iodo.

The term “acrylic polymer(s)” as used herein means 1) alkyl methacrylate homo polymers, 2) copolymers of alkyl methacrylates with other alkyl methacrylates or alkyl acrylates or other ethylenically unsaturated monomers, 3) alkyl acrylate homo polymers, and 4) copolymers of alkyl acrylates with other alkyl acrylates or alkyl methacrylates or other ethylenically unsaturated monomers

The components of the present invention are described below. Also included are non-limiting descriptions of the optional and preferred embodiments useful in the formulation of the present invention.

In one aspect of the invention, the compositions of the invention comprise a polymer. The polymer can have a molecular weight that is greater than about 2,000 daltons (Da), and more preferably is greater than about 5,000 Da. In another aspect of the invention, the polymer has a molecular weight of about 10,000 Da to about 250,000 Da. Thus, the ranges of molecular weights for the polymer can be from about 2,000 Da to about 200,000 Da, preferably about 5,000 Da to about 150,000 Da, more preferably about 7,000 Da to about 150,000 Da, or from about 10,000 Da to about 80,000 Da. The polymer can have an average molecular weight of about 5,000 Da, about 7,000 Da, about 10,000, about 15,000 Da, about 17,500 Da, about 20,000 Da, about 30,000 Da, about 35,000 Da, about 40,000 Da, about 50,000 Da, and about 60,000 Da are particularly preferred.

Non-limiting examples of polymers for use in the present invention include: polyesters, polyethers, poly(orthoesters), poly(vinyl alcohols), polyamides, polycarbonates, polyacrylamides, polyalkylene glycols, polyalkylene oxides, polyalkylene terephthalates, polyolefins, polyvinyl ethers, polyvinyl esters, polyvinyl halides, polyvinylpyrrolidone, polyglycolides, polysiloxanes, polylactides, polyurethanes, polyethylenes, polyanhydrides, polyhydroxyacids, polypropylfumerates, polycaprolactones, polyacetals, polyurethanes, polyphosphazenes, polyacrylates, polymethacrylates, polycyanoacrylates, polyureas, polystyrenes, polyamines, olefinic polymers derived from ROMP reaction with functionalized monomers, and block- or co-polymers thereof.

In one aspect of the invention, the polymer is an acrylic polymer, preferably a methyacrylate. Preferably, the (meth)acrylic copolymers according to the present invention comprise about 80 to about.99.8 parts by weight of alkyl acrylic acid ester monomers having from about 2 to about 14 carbon atoms of alkyl group.

The above alkyl acrylic acid ester monomers having 2 about 14 carbon atoms of alkyl group may be ethyl(meth)acrylate, n-propyl(meth)acrylate, isopropyl(meth)acrylate, n-butyl(meth)acrylate, t-butyl(meth)acrylate, sec-butyl(meth)acrylate, pentyl(meth)acrylate, 2-ethylhexyl(meth)acrylate, n-octyl(meth)acrylate, isooctyl(meth)acrylate, isononyl(meth)acrylate, lauryl(meth)acrylate, and tetradecyl(meth)acrylate.

Specific examples of acrylate polymers comprise monomers including alkyl acrylates such as methyl acrylate, ethyl acrylate, propyl acrylate, butyl acrylate, pentyl acrylate, hexyl acrylate, 2-ethylhexyl acrylate, octyl acrylate, nonyl acrylate, decyl acrylate and dodecyl acrylate; alkoxyalkyl acrylates such as methoxyethyl acrylate, ethoxyethyl acrylate, propoxyethyl acrylate, butoxyethyl acrylate and ethoxypropyl acrylate; alkyl methacrylates such as methyl methacrylate, ethyl methacrylate, propyl methacrylate, butyl methacrylate, pentyl methacrylate, hexyl methacrylate, 2-ethylhexyl methacrylate, octyl methacrylate, nonyl methacrylate, decyl methacrylate and dodecyl methacrylate; alkoxyalkyl methacrylates such as methoxyethyl methacrylate, ethoxyethyl methacrylate, propoxyethyl methacrylate, butoxyethyl methacrylate and ethoxypropyl methacrylate; (poly)alkylene glycol diacrylates such as ethylene glycol diacrylate, diethylene glycol diacrylate, triethylene glycol diacrylate, poly(ethylene glycol)diacrylate, propylene glycol diacrylate, dipropylene glycol diacrylate and tripropylene glycol diacrylate; polyvalent acrylates such as trimethylolpropane triacrylate; polyvalent methacrylates such as trimethylolpropane trimethacrylate; acrylates of alicyclic alcohols such as cyclohexyl acrylate; methacrylates of alicyclic alcohols such as cyclohexyl methacrylate; and fluorine-substituted alkyl methacrylates and fluorine-substituted alkyl acrylates.

These acrylate monomers can be used alone or in combination. These acrylate monomers may be liquid, solid, or gaseous under the reaction conditions. For ease of operation, liquid monomers are preferably used in reactions.

An acrylic polymer according to the present invention can be formed by the copolymerization of acrylic monomers described above. The acrylic monomers can have functional groups so that the acrylic monomers can react with a cross-linker. Alternatively, the acrylic polymer of the present invention can be obtained from a commercial source.

Commercially available examples of acrylic polymers for use in the present invention include, but are not limited to, ELVACITE® 2041, ELVACITE® 2014 ELVACITE® 2552, and ELVACITE® 2552C which can be obtained from Lucite International, Inc. Texas and INEOS Acrylics of Cordova, Tenn.; and Br85, manufactured by Dianal America, Inc. of Pasadena, Tex.

In another aspect of the invention, the composition comprises dissolving the acrylic polymer in an organic solvent. The organic solvent can be any organic solvent, such as for example, methyl acetate, ethyl acetate, propyl acetate, isopropyl actate, butyl acetate, isobutyl acetate, tert-butyl acetate, pentane, hexane, heptane, acetone, methyl ethyl ketone, xylene, and the like, and combinations thereof.

In one aspect of the invention, the organic solvent is an alkyl ester. The alkyl group can have from one to fourteen carbon. Examples of alkyl group include, but are not limited to, methyl, ethyl, n-propyl, isopropyl, isobutyl, sec-butyl, tert-butyl, pentyl, n-hexyl, octyl, dodecyl, and the like. The acid can be acetic acid, benzoic acid, butyric acid, capric acid, caproic acid, caprylic acid, citric acid, formic acid, fumaric acid, gluconic acid, glyceric acid, glycolic acid, heptanoic acid, lactic acid, lauric acid, linoleic acid, maleic acid, malic acid, myristic acid, nonanoic acid, oleic acid, palmitic acid, propionic acid, salicyclic acid, sorbic acid, stearic acid, tartaric acid, undacanoic acid, undecylenic acid and valeric acid. Preferably, the acid can be acetic acid, citric acid, lactic acid or lauric acid.

The compositions of the invention comprise the acrylic polymer dissolved in the solvent system in a ration of about 1:100 to about 100:1 by weight. The ratio of the polymer to the solvent can be, for example, 1:50, about 1:40, about 1:30, about 1:20, about 1:15, about 1:12, about 1:10, about 1:8, about 1:7, about 1:5, and the like.

The nail polish compositions of the invention can be formulated as a clear, colorless lacquer component, optionally mixed with a pigment component when a colored polish is desired. The lacquer component comprises the acrylic polymer dissolved in an organic solvent. drying accelerator, film formers, as well as plasticizers and optionally a thixotropic agent, a brightening agent, a plasticizer, and aromatic ketones. The nail polish composition can comprise the acrylic polymer dissolved in an organic solvent, pigments, film former, a brightening agent, thixotropic agent, plasticizer, aromatic ketones, and solvents.

In one aspect of the invention, the nail polish lacquer can include about 0.1% to about 60%, preferably about 0.2% to about 20%, more preferably about 0.5% to about 10% of the acrylic polymer dissolved in an organic solvent; about 5% to about 40%, preferably about 4% to about 20%, more preferably about 7% to about 17% by weight, and most preferably about 14.4% of a film-forming polymer; about 0.1% to about 10%, preferably about 0.2% to about 8%, and most preferably about 2.5% to about 3.5% by weight of a plasticizer; about 5% to about 80% of a solvent system, preferably about 45% to about 55% of the solvent system. The nail polish lacquer of the invention can optionally contain a gelling agent, such as about 0.2% to about 0.4% of least one thixotropic agent, a drying accelerator, a suspension agent, a UV inhibitor, pigments and dyes, and the like wherein the above percentages are based on the total weight of the lacquer component. Most preferably, the final fingernail lacquer contains from about 0.5 to about 10 weight percent of the acrylic co-polymer.

The invention is directed to a nail lacquer or topcoat composition comprising:

| | Weight % |
|---|---|
| Nitrocellulose or CAB | 4-20% |
| Acrylic Polymer or | .5-10% |
| Copolymer | |
| Toluene sulfonamide | 1-10% |
| Formaldehyde resin | |
| Or other modifiers for | |
| shine and flexibility | |
| Plasticizer | 1-8% |
| Solvents and diluents | 10-50% |
| UV inhibitor | .01-1% |
| Suspension agent for | .25-1% |
| Nail Lacquer only | |
| Pigments and dyes for | .1-5% |
| Nail Lacquer only | |

The preferred embodiment of the invention is a Fingernail Lacquer composition comprising:

| | Weight % | |
|---|---|---|
| Nitrocellulose or CAB | 14.4% | (4.20%) |
| Acrylic Copolymer | 3.5% | (.5-10%) |
| Elvacite 2552C | | |
| Toluene sulfonamide | 2.9% | (1-10%) |
| Formaldehyde Resin | | |
| Acetyl Tributyl Citrate | 2.9% | (1-8%) |
| Butyl Acetate | 34.32% | (10-50%) |
| Ethyl Acetate | 39.13% | (10-50%) |
| Etocrylene | .028% | (.01-1%) |
| Pigments or Dyes | 3% | (.1-5%) |

The preferred embodiment of the invention is also a Fingernail Topcoat composition comprising:

| | Weight % | |
|---|---|---|
| Nitrocellulose or CAB | 15% | (4-20%) |
| Acrylic Copolymer | 3.5% | (.5-10%) |
| Elvacite 2552C | | |
| Toluene sulfonamide | 3% | (1-10%) |
| Formaldehyde Resin | | |
| Acetyl Tributyl Citrate | 3% | (1-8%) |
| Butyl Acetate | 35.23% | (10-50%) |
| Ethyl Acetate | 40.24% | (10-50%) |
| Etocrylene | .03% | (.1-1%) |

The composition of the present invention is made using conventional formulation and mixing techniques. The original formulation was composed by mixing 35 weight percent Lucite Elvacite Resin 2552C with 65% Ethyl Acetate and adding the resulting mixture to a generic fingernail topcoat or fingernail lacquer to create a mixture that contains from 0.5 to 10 weight percent co-polymer.

The compositions of the invention preferably contain polar aprotic solvents, such as, for example, esters, preferably ethyl acetate, N,N-dimethylformamide, xylene, and the like. Preferably, the compositions do not contain substantial amounts of a solvent or an excipient that is highly polar and/or protic. For example, solvents with polarities that approach water preferably are not included in the compositions of the invention. Such solvents, include, for example alcohols, such as methanol, ethanol, propanol, isopropanol, butanol, isobutanol, tert-butanol, and the like. Preferably, the compositions contain an alcohol that is less than about 5% by total weight, less than about 7% by total weight, less than about 8% by total weight, less than about 9% by total weight, less than about 10% by total weight, less than about 11% by total weight, less than about 12% by total weight, and the like. As one of skill in the art will appreciate, the higher concentration of an alcohol can result in the acrylic polymer, such as Elvacite 2552 or Elvacite 2552C to precipitate from the composition and the resultant composition can appear cloudy.

The use of the nail lacquer and fingernail topcoat is as follows: The fingernail is thoroughly cleaned to remove all oil from the nail.

A base-coat nail lacquer is applied to the fingernail to promote proper adhesion of the nail lacquer to the fingernail. Wait until the base-coat has properly dried before applying the following fingernail lacquer coat. Fingernail lacquer is applied to the fingernail over the base coat. Two coats may be applied for some colors for even color distribution. Wait until the fingernail lacquer is properly dry before applying the next coat. One or two layers of fingernail top coat are applied over the top of the fingernail lacquer. Wait until the first coat is properly dry before applying the next coat.

The lacquer of the invention, when applied to the nail, preferably dry in an ambient atmosphere is less than about 7 minutes, preferably in less than about 5 minutes, more preferably in less than about 3 minutes. The composition of the invention are durable and typically do not chip or crack for a period of at least about three days of wearing on a human or synthetic nail. Coatings formed of the lacquer component per se, as well as the coatings formed from compositions which include both the lacquer and pigment components, exhibit an acceptable gloss. The lacquer of the invention, when applied to the nail, preferably last for at least about a week, preferably for at least about two weeks, more preferably for at least about three weeks. The lacquer can then be reapplied, or the previous layer(s) can be removed, and new layer applied.

The lacquer of the invention can be removed from nails using standard nail polish removers or an organic solvent, such as acetone.

### Alternative Ways to Make this Formulation

There are other alternative ways that the fast drying topcoat or nail lacquer that is long lasting, has a high gloss, and can be removed by nail polish remover can be created:

An alternative formulation would be to replace the Elvacite 2552C resin with an Another Film Forming Resin that has similar properties and is functionally equivalent to the Elvacite 2552C resin. These can be film-forming water-insoluble polymers selected from the group consisting of polyurethanes, polyacryls, polymethacryls, polystyrene-polyacryl mixtures, styrene-acryl copolymers, siloxane-urethane copolymers, cellulosic polymers, polyesters, polyurethane-polymethacryl mixtures, silicone-acryl copolymers, vinyl acetate polymers, and mixtures thereof.

Another alternative formulation would be to replace the Elvacite 2552C resin with Another Acrylic or Acrylic Copolymer Resin that has similar properties and is functionally equivalent to the Elvacite 2552C resin. These can be film-forming water-insoluble polymers selected from the group consisting of, polyacryls, polymethacryls, polystyrene-polyacryl mixtures, styrene-acryl copolymers, and mixtures thereof.

Another alternative formulation would be to use alternative plastisers. These would be selected from the group consisting of dibutyl phthalate, Camphor, dioctyl phthalate, castor oil, tricresyl phophate, butyl phthalate, butyl blycolate, triphenal phosphate, glyceryl tribenzoate benzyl benzoate, butyl stearate, triethyl citrate, propylene glycol adipate and other common plastisizers used in acrylic and nitrocellulose coatings for flexibility and chip resistance.

Another alternative formulation would be to change the solvent formulation using any of the other compatible solvents with the current acrylic and nitrocellulose or CAB film forming components. These would include heptane, methyl ethyl ketone, amyl acetate, acetone, mono-N-butyl ether, light petroleum napthas, and so on.

Another embodiment of the invention would be to add glitter to the fingernail lacquer or fingernail topcoat formulation to form a fingernail glitter that has the same properties of new fingernail lacquer or fingernail topcoat formulation.

Another embodiment of the invention would be to add nonessential components such as preservatives, vitamins, herbal extracts, FDC acceptable dyes, moisturizing agents and so on.

In another aspect of the invention, the compositions can optional contain aromatic ketones. Exemplary aromatic ketones useful in the pigment component include 2,2-dimethoxy-2-phenyl acetophenone, 1-hydroxy cyclohexyl phenyl ketone, 2-hydroxy-2-methyl-1-phenyl-propane-1-one and 2,4,6-trimethyl benzoldiphenyl.

All printed patents and publications referred to in this application are hereby incorporated herein in their entirety by this reference. While the preferred embodiment of the invention has been illustrated and described, it will be appreciated that various changes can be made therein without departing from the spirit and scope of the invention.

What is claimed is:

1. A nail polish composition comprising:
about 0.5% to about 10% of an acrylic polymer dissolved in an organic solvent;
about 4% to about 20% by weight of a film-forming polymer, wherein the film-forming polymer is nitrocellulose, cellulose acetate, cellulose acetate-butyrate, ethyl cellulose, polyvinyl acetate, polyvinyl butyrate, or combinations thereof;
about 1% to about 4% by weight of a plasticizer; and
about 10% to about 50% by weight of a solvent system.

2. The nail polish composition of claim 1, the composition further comprising about 0.25% to about 1% of a suspension agent.

3. The nail polish composition of claim 1, the composition further comprising about 0.1% to about 5% of a pigment, a dye or a combination thereof.

4. The nail polish composition of claim 1, wherein the acrylic polymer comprises alkyl acrylate monomers.

5. The nail polish composition of claim 4, wherein the acrylic polymer is Elvacite 2552C or Elvacite 2552.

6. The nail polish composition of claim 1, wherein the plasticizer is toluene sulfonamide, formaldehyde resin, camphor, a phthalate, a phosphate, a benzoate, or combination thereof.

7. The nail polish composition of claim 1, wherein the solvent system comprises methyl acetate, ethyl acetate, butyl acetate, hexane, heptane, methyl ethyl ketone, acetone, or combinations thereof.

8. A fingernail coating comprised of:

| | Weight % |
|---|---|
| Nitrocellulose or CAB | 4-20% |
| Acrylic Polymer or | .5-10% |
| Copolymer | |
| Toluene sulfonamide | 1-10% |
| Formaldehyde resin | |
| Or other modifiers for | |
| shine and flexibility | |
| Plasticizer | 1-8% |
| Solvents and diluents | 10-50% |
| UV inhibitor | .025-1% |
| Suspension agent for | .25-1% |
| Nail Lacquer only | |
| Pigments and dyes for | .1-5% |
| Nail Lacquer only | |

9. A fingernail Lacquer composition comprising:

| | Weight % |
|---|---|
| Nitrocellulose or CAB | 4-20% |
| Acrylic Copolymer | .5-10% |
| Elvacite 2552C | |
| Toluene sulfonamide | 1-10% |
| Formaldehyde Resin | |
| Acetyl Tributyl Citrate | 1-8% |
| Butyl Acetate | 10-50% |
| Ethyl Acetate | 10-50% |
| Etocrylene | .1-1% |
| Pigments or Dyes | .1-5% |

10. A fingernail Topcoat composition comprising:

| | Weight % |
|---|---|
| Nitrocellulose or CAB | 4-20% |
| Acrylic Copolymer | .5-10% |
| Elvacite2552C | |
| Toluene sulfonamide | 1-10% |
| Formaldehyde Resin | |
| Acetyl Tributyl Citrate | 1-8% |
| Butyl Acetate | 10-50% |
| Ethyl Acetate | 10-50% |
| Etocrylene | .01-1.0% |

11. The composition of claim 9 where the acrylic polymer or copolymer is Elvacite 2552C or Elvacite 2552.

12. A method for increasing the longevity of a nail enamel by applying the composition of claim 9, wherein the composition is reapplied after about one to about three weeks.

13. A method for decreasing the time to dry all coats of fingernail lacquer or polish, the method comprising applying the fingernail coating of claim 9 and drying the fingernail under ambient conditions for a drying time, wherein the drying time is less than about three minutes.

14. A method to have a high shine nail coating by applying the composition of claim 9.

15. A method of removing the composition of claim 9, the method comprising applying acetone or a fingernail polish remover to the surface containing the composition.

16. A method of hardening the composition of claim 9, the method comprising applying the composition to fingernails and air drying.
